(19) United States
(12) Patent Application Publication
(10) Pub. No.: US 2025/0382562 A1
(43) Pub. Date: Dec. 18, 2025

(54) Magnetic Resonance radio frequency coils for imaging and spectroscopy of tissue cultures, organoids, and lab on a chip

(71) Applicant: Debra Strick Rivera, Topanga, CA (US)

(72) Inventor: Debra Strick Rivera, Topanga, CA (US)

(21) Appl. No.: 18/742,469

(22) Filed: Jun. 13, 2024

(51) Int. Cl. C12M 1/34; C12M 3/06

(52) U.S. Cl. CPC C12M 41/36; C12M 23/16

(57) ABSTRACT

The present invention provides a device consisting of one or more radio frequency coils for obtaining magnetic resonance imaging or spectra from a cell culture platform in which the radio frequency coils are either wired or wireless. In one embodiment, a multichannel receive array of coil elements is designed such that the wells of the standard 24-well plate sits nested in the holder that holds the array, and optionally, a transmit coil. In another embodiment, a micro-solenoid is incorporated around the microfluidics channel of a lab on a chip. In either implementation, the receive elements can be either solenoids, single-loop or saddle coil configuration. The device may also be designed in such a way to allow for compatibility with an electrode array.

### CROSS REFERENCE TO RELATED APPLICATION

Not Applicable

### STATEMENT REGARDING FEDERALLY SPONSORED RESEARCH OR DEVELOPMENT

Not Applicable

### BACKGROUND

The present invention provides a device consisting of one or more radio frequency coils for obtaining magnetic resonance imaging or spectra from a cell culture platform in which the radio frequency coils are either wired or wireless.

Nuclear Magnetic Resonance (NMR) and Magnetic Resonance Imaging (MRI) and spectroscopy (MRS) are non-destructive means of interrogating and analyzing samples. By integrating radio frequency coils tuned to the frequency or frequencies of interest for NMR/MRI/MRS, the powerful imaging and spectroscopic capabilities can be added to standard microfluidics and cell culturing platforms including organelles.

U.S. Pat. No. 10,954,482B provides integrated Organ-on-Chip micro-physiological systems representations of living Organs and support structures for such micro-physiological systems. While the cited prior art provides methods for the analysis of drug efficacy, toxicity, pharmacokinetics, and pharmacodynamics using the integrated micro-physiological system instrumentation that consisting of multiple individual Organ Chips coupled together micro-fluidically, the present invention provides a device consisting of one or more radio frequency coils for obtaining magnetic resonance imaging or spectra from a cell culture platform in which the radio frequency coils are either wired or wireless.

The present invention is an improvement in the existing art and the person skilled in the art will be able to notice the same, allowing for non-destructive means of analyzing cell cultures in a manner that can also be translated non-invasively in vivo.

### BRIEF DESCRIPTION OF THE INVENTION

In one embodiment, a multichannel receive array of coil elements (3) is designed such that the wells of the standard 24-well plate sits nested in the holder (1) that holds the array, and optionally, a transmit coil (2).

In another embodiment, a micro-solenoid (5), is incorporated around the microfluidics channel of a lab on a chip. In either implementation, the receive elements can be either solenoids, single-loop, saddle coil configuration. Decoupling circuitry can be employed if needed to decouple neighboring elements of a multi-element array. The elements can also be designed to be stable in water for a period of time, if the cell culture set up requires it, or space constraints make it beneficial to do so. To aid fabrication of the microfluidics integrated implementation, holes for wire to be laced through as a solenoid can be incorporated into the design of the lab-on-a-chip substrate to control the spacing of the turns.

For example, a solenoid integrated around the so called “cell channel bilayer” mentioned in [U.S. Pat. No. 10,954,482B2], would allow for imaging and spectroscopy of the organ-on-a-chip platform. In a further embodiment, the radio frequency coil array can be placed under a petri dish containing for example, organoid in Matrigel, such that one set of coils is used for localizing the cells clusters, and another set of coils is used for deriving spectra from the different clusters. A further embodiment can include a static magnet, while another embodiment can include spatial encoding gradients.

In another embodiment, the RF coil can be implemented in such as manner such that it is also possible to use an electrode, electrode array, microelectrode array, or high-density microelectrode array. The electrode area such as in Le Floch et al [10.1002/adma.202106829] can be used as is or redesigned such that the electrode is made of materials matched to the magnetic susceptibility of tissue, including but not limited to gold, copper coated in gold, or an alloy of platinum and gold. The RF coil or coils can be integrated into the su-8 ring or lid structure, or built around the wells of a well plate. The coil can be built into the organoid hosting volume of the microelectrode containing unit or inserted only for imaging and spectroscopy purposes.

One use of such a system is to non-destructively interrogate the structure and function of the interactions and characteristics of the system. The invention is motivated by the potential to derive information from tumor cells and in turn apply that knowledge in vivo. While tumor organoids are already being used to identify candidate treatments for a given patient, by combining RF coils into the infrastructure of those experiments, individualized biomarkers of treatment response and the influence of pH can be explored in situ and utilized in vivo using existing MRI methods [10.3390/metabo12050409].

### BRIEF DESCRIPTION OF THE DRAWINGS

FIG. 1 demonstrates a microfluidics system characteristic of an organ on a chip model, comprising a 1 microfluidics substrate, 2 well for introducing cells, wells for introducing media and toxicant 3, and a 4 cell channel bilayer. A microsolenoid 5, shown in the inset, is intended to surround the cell channel bilayer 5. An example dataset 6 of pure water and mayonnaise collected by the microcoil 5.

FIG. 2 demonstrates a microfluidics system with a microfluidics substrate 1 and a cell channel bilayer 4, with a microsolenoid 5 wrapped around.

FIG. 3 demonstrates an imaging array for a standard 24-well plate.

### DETAILED DESCRIPTION OF THE DRAWINGS

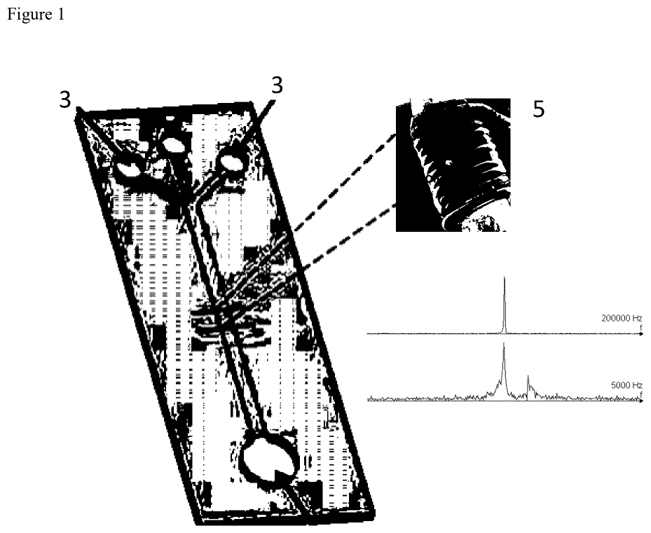

FIG. 1 demonstrates a microfluidics system characteristic of an organ on a chip model, comprising a 1 microfluidics substrate, 2 well for introducing cells, wells for introducing media and toxicant 3, and a 4 cell channel bilayer. A microsolenoid 5, shown in the inset, is intended to surround the cell channel bilayer 5. An example dataset 6 of pure water and mayonnaise collected by the microcoil 5.

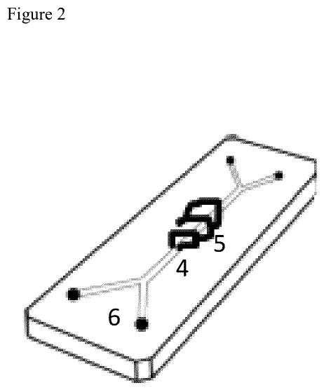

FIG. 2 demonstrates a microfluidics system with a microfluidics substrate 1 and a cell channel bilayer 4, with a microsolenoid 5 wrapped around.

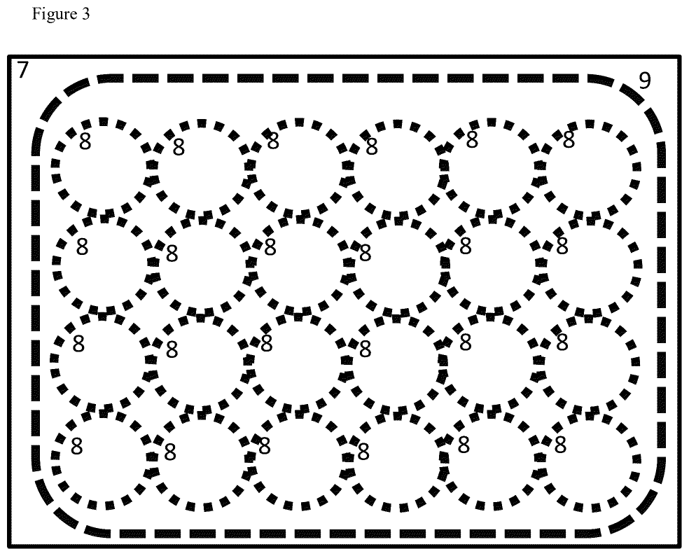

FIG. 3 demonstrates an rf coil system for analyzing a standard 24-well plate, comprising of a substrate 7, a receive array made up of receive elements 8, and a transmit coil 9.

What is claimed for is:

1. A device consisting of one or more radio frequency coils for obtaining magnetic resonance imaging or spectra from a cell culture platform in which the radio frequency coils are either wired or wireless.

2. A device such as in claim 1 wherein the cell culture platform is a well plate.

3. A device such as in claim 2 wherein one radio frequency coil is used per well.

4. A device such as in claim 3 wherein the radio frequency coil elements are submerged in the wells.

5. A device such as in claim 3 wherein the well plate is placed onto the coil former.

6. A device such as in claim 1 wherein a plurality of cell cultures with radio frequency coils can be placed in a static magnet simultaneously.

7. A device such as in claim 1 wherein a static magnetic field generating system is included in the design.

8. A device such as in claim 1 wherein one or more magnetic field gradient systems are included in the design.

9. A device such as in claim 1 wherein the cell culture platform is a microfluidics device.

10. A device such as in claim 1 wherein the microfluidics device is a lab on a chip or organ on a chip.

11. A device wherein an radio frequency coil for magnetic resonance imaging or nuclear magnetic resonance spectroscopy is incorporated on a microfluidics device in which the radio frequency coils are either wired or wireless.

12. A device as in claim 11 where holes are made in the microfluidics substrate to accommodate the wire of the radio frequency coil.

13. A device as in claim 1 in which a microelectrode array is incorporated into the set up.
